(19) United States
(12) Patent Application Publication
(10) Pub. No.: US 2023/0145643 A1
(43) Pub. Date: May 11, 2023

(54) AGENTS FOR USE IN THE THERAPEUTIC OR PROPHYLACTIC TREATMENT OF RETINAL PIGMENT EPITHELIUM ASSOCIATED DISEASES

(71) Applicants: Songhomitra PANDA-JONAS, Weinheim (DE); Jost JONAS, Heidelberg (DE)

(72) Inventors: Songhomitra PANDA-JONAS, Weinheim (DE); Jost JONAS, Heidelberg (DE)

(21) Appl. No.: 17/915,334

(22) PCT Filed: Mar. 31, 2021
(86) PCT No.: PCT/EP2021/058500

§ 371 Date: Sep. 28, 2022

(30) Foreign Application Priority Data
Mar. 31, 2020 (EP) 20167033.8

(51) Int. Cl. A61K 38/18; A61P 27/02; A61K 9/00

(52) U.S. Cl. CPC A61K 38/1808; A61P 27/02; A61K 9/0048

(57) ABSTRACT

The present invention relates to a pharmaceutical composition comprising an epidermal growth factor receptor (EGFR) agonist for use in the treatment of a retinal pigment epithelium (RPE) damage associated disease in a patient, wherein the EGFR agonist comprises the EGF family consensus amino acid sequence CX₇CX₄₋₅CX₁₀₋₁₃CXCX₅GXRC (SEQ ID NO: 1), wherein X is any proteogenic amino acid, wherein the RPE damage associated disease is selected from age-related macular degeneration (AMD), retinitis pigmentosa, cone-rod dystrophies or cone dystrophies, polypoidal choroidal vasculopathy, and Stargardt's disease.

### FIELD OF THE INVENTION

The present invention relates to diseases associated with the retinal pigment epithelium (RPE) as well as products and methods in the treatment and prophylaxis of the diseases.

### BACKGROUND OF THE INVENTION

Age-related macular degeneration (AMD) is a disease of the central region of the retina, called macula (FIG. 1). It affects central visual acuity and can lead to the inability to read or to recognize faces and it can even lead to blindness. Due to its strongly age-related prevalence and due to the aging of the population, AMD has become one of the most common causes of irreversible visual impairment and blindness worldwide, in particular in the high-income countries.

In its early stage, AMD is morphologically characterized by accumulation of material beneath the retinal pigment epithelium (RPE), or between the RPE and the retinal photoreceptor outer segments, in the form of so-called drusen or subretinal drusenoid deposits in the macular region. The RPE is a monolayer of pigmented cells enclosing the retinal photoreceptor outer segments and being deeply involved in functions such as the regeneration of the photo pigments, phagocytosis of the photoreceptor outer segments and other processes that are essential for the function and survival of the retinal photoreceptors (FIG. 2).

The early stage of AMD with small or medium-large drusen is usually followed or accompanied by the development of irregularities of the RPE, which shows a focal hyperpigmentation and hypopigmentation. Eventually, the RPE can get completely depigmented or lost in the form of so-called geographic atrophy, in which due to the changes or loss of RPE cells, the retinal photoreceptors can no longer function. The region of geographic atrophy subsequently corresponds to an absolute scotoma in the visual field. Since geographic atrophy affects the central and pericentral region, it is associated with a pronounced loss in visual acuity and reading ability up to legal blindness.

Besides of, and parallel to geographic atrophy, a choroidal neovascularization can develop in the late stage of AMD. It consists of newly formed blood vessels usually starting from the choroid, breaking through Bruch's membrane as the border between the RPE and the choroid, and invading the space beneath the RPE or the space on top of the RPE directly under retina. It eventually leads to the formation of a scar in the center of the retina with destruction of the retinal architecture. The neovascularization, also referred to as “wet AMD”, can develop also independently of a pre-existing geographic atrophy, the late stage of the above described so-called “dry AMD”. The risk factors for the development of AMD have intensively been explored and the list of proven or potential risk factors includes parameters such as older age, axial hyperopia, smoking, male gender, arterial hypertension, hyperlipidemia, chronic kidney disease, hepatitis B surface antigen positivity, liver cancer, coronary heart disease, lower education levels and increased serum white blood cell levels.

Despite of the intensive research carried out in the last 50 years, the etiology of AMD has remained mostly elusive so far. Genes that have been found to be associated with AMD include those connected with the complement factor system (e.g., CFH rs10737680, CFI rs4698775) and others (such as ARMS2, HTRA1, rs10490924, CETP rs3764261, ADAMTS9, rs6795735, C2e CFB rs429608, TGFBR1 rs334353, APOE rs4420638, and VEGFA rs943080).

Already at its earliest stage, the development and progression of AMD up to its final stage is intimately associated with changes of the RPE. One may therefore assume that the RPE is the primary structure, or one of the structures first affected, in the chain of pathogenic events leading subsequently to the late stage of AMD including geographic atrophy and choroidal neovascularization. This assumption is supported by the high photonic and heat exposure of the RPE in the center of the macula where most of the incoming light is focused on a small spot. Lifelong, the optical system of the eye with a total refractive power of about 50 to 60 diopters bundles most of the incoming light onto the tiny spot of the retinal center. Most of the light, which is not absorbed by the photoreceptor outer segments, gets absorbed as stray light by the RPE. The heat generated by the focused light in the macular RPE is cooled down by the choroidal blood stream, which is separated from the RPE by Bruch's membrane. The latter consists of two basal membranes separated by collagenous and elastin layers. While the photoreceptor outer segments are renewed approximately every 10 to 14 days, the regenerative capacity of the RPE, if present at all, is considerably lower. It is therefore likely that after decades of light and heat exposure, light-associated and heat-associated degenerative changes may develop in the RPE.

Damage of the RPE is also associated with other disease such as retinitis pigmentosa. Retinitis pigmentosa is a group of genetic diseases in which due to a mutation in one of 50 or more genes, proteins associated with the photoreceptor inner or outer segments or with other parts of the visual cycle get misfolded or changed in their structure. As a consequence, the RPE decreases in its ability to orderly phagocytose the outer photoreceptor segment discs so that the outer photoreceptor segment debris accumulates and the RPE gets damaged. In the further course of the disease, the RPE transforms and proliferates into the retina while it no longer supports the photoreceptors in the visual process. Ultimately, the RPE and photoreceptors get mostly lost and blindness results.

In other dystrophic and degenerative diseases of the retina, RPE and choroid such as cone-rod dystrophies or cone dystrophies, polypoidal choroidal vasculopathy, and Stargardt's disease, the RPE is secondarily or primarily involved and undergoes extensive degenerative changes up to its complete loss.

Cone dystrophy and cone-rod dystrophies are inherited disorders of the retinal photoreceptors characterized by the loss of cone photoreceptors, and cone and rod photoreceptors, respectively. The RPE is secondarily involved, gets damaged and eventually lost.

Polypoidal choroidal vasculopathy belongs to a spectrum of pachychoroidal disorders in which an abnormally thick choroid is associated with a serosanguineous detachment of the RPE and subretinal choroidal neovascularization with secondary scar formation. The RPE is secondarily involved in the course of the disease.

Stargardt's disease is an inherited single-gene retinal disorder usually caused by mutations in the ABCA4 gene, resulting in a malfunction of the ATP-binding cassette transporter (ABCA4) protein, a part of the visual phototransduction cycle. As a sequel, movement of vitamin A throughout the retina is reduced, leading to an accelerated formation of toxic vitamin A dimers and associated degradation byproducts. Damaging the retinal cells, these molecules lead to an accumulation of lipofuscin in the RPE and further RPE damage.

For these RPE associated diseases, to date very little treatment forms are available. One of the reasons is that observation of and experimentation with the RPE is difficult in vivo due to location at the back of the eye and the blood-retinal barrier, which makes drug treatments from passing from the blood to the RPE difficult.

For example, while AMD research has been conducted for many years, there is still no available chemical or surgical treatment for reversal of vision loss in the dry form of the disease (Gagliardi et al. 2019; Kandasamy et al. 2018; Laude et al. 2020; Mehta et al. 2018; Nazari et al. 2015; Singh et al. 2001).

Many researchers are exploring possibilities for treatment, such as slowing deterioration in the intermediate stage through high doses of vitamin and nutrient supplements such as lutein and zeaxanthin (Donaldson et al. 2006), treatments like laser photocoagulation (Tode et al. 2019), use of stem cells or other cells to replace degraded cells, (Binder et al. 2004; Yuan et al. 2015; Hu et al. 2015) and even gene therapy (Peng et al. 2011, Koirala et al. 2013).

It is noted that there is often a nutritional therapy prescribed for dry AMD, in the early stage just a healthy diet high in antioxidants, and in further advanced but still dry AMD, supplements to add higher quantities of certain vitamins, minerals and other nutrients which are supposed to increase healthy pigments and support cell structure. However, the nutritional therapy can often neither improve the condition nor stop the progress of the AMD. It may have an effect on the speed of the progress, but even this is under debate (Merle et al. 2019; Vavvas et al. 2018).

For the “Wet AMD”, there is a treatment based on anti-VEGF drugs. Vascular endothelial growth factor's (VEGF) normal function is to create new blood vessels during embryonic development, new blood vessels after injury, new vessels in the muscle following exercise, and new vessels (collateral circulation) to bypass blocked vessels (Cooper et al. 1999). In the case of macular disease, VEGF promotes the growth of new, weak blood vessels from the choroid through Bruch's membrane into the space beneath the RPE and/or into the subretinal space, and those vessels leak blood, lipids, and serum into the subretinal space and into the retinal layers. Anti-VEGF drugs include VEGF antibodies, such as ranibizumab (Lucentis® from Novartis), Bevacizumab (Avastin® from Genentech/Roche) and VEGF binding fusion proteins such aflibercept (Eylea® from Regeneron/Bayer) and aptamers, such as Pegaptanib (Macugen® from Pfizer).

Anti-VEGF drugs are reported to make the newly formed blood vessels to recede or to obliterate, to slow down the progression of the disease, and, in some cases, to lead to a moderate gain in vision. The anti-VEGF agents have no effect on the RPE and therefore, the symptoms caused by the geographic atrophy (dry AMD).

Therefore, there is a need for an effective treatment of dry AMD and other RPE damage associated diseases.

### SUMMARY OF THE INVENTION

The present invention is, inter alia, based on the surprising finding that dry AMD can be effectively treated by increasing the effect of a growth factor, i.e. an activating agonist of the epidermal growth factor receptor (EGFR). In particular, the inventors were able to show that intravitreal application of EGF into the eye of patients with geographic atrophy as the late stage of dry AMD showed an improvement in visual function and macular morphology. Moreover, subjectively, the patients noted an improvement in vision with higher clarity and less visual distortion.

When investigating the molecular mechanism underlying this therapeutic effect, the inventors were able to identify that addition of EGF has a promoting effect on migration and proliferation of cultured RPE cells. Based on this finding, it is hypothesized that functional improvement and proliferation and migration of the RPE cells promote a repair of the damaged RPE layer, which in turn leads to the reduction of dry AMD in the treated patient and an amelioration of the patient's sight. Moreover, it was confirmed that the EGF effect is mediated via the EGFR PI3K/AKT signaling pathway.

Therefore, the present invention relates to a pharmaceutical composition comprising an EGFR agonist for use in the treatment of an RPE damage associated disease in a patient, wherein the EGFR agonist comprises the EGF family consensus amino acid sequence CX₇CX₄₋₅CX₁₀₋₁₃CXCX₅GXRC (SEQ ID NO: 1), wherein X is any proteogenic amino acid, wherein the RPE damage associated disease is selected from AMD, retinitis pigmentosa, cone-rod dystrophies or cone dystrophies, polypoidal choroidal vasculopathy, and Stargardt's disease.

### FIGURES

FIG. 1 shows the schematic anatomy scheme of the eye

FIG. 2 the histological anatomy of the retina, retinal pigment epithelium, and choroid

FIG. 3 shows an optical coherence tomographic image of the macular region, i.e. the fovea, of an eye with geographic atrophy, with a tissue thickness of 69 μm at baseline prior to the injection (FIG. 3a) and the optical coherence tomographic image of the same region after the injection

FIG. 4 shows the perimetric results at baseline with a relative central scotoma before injection (FIG. 4a; black arrow) and at one week after the injection with an improvement of the central visual field (FIG. 4b)

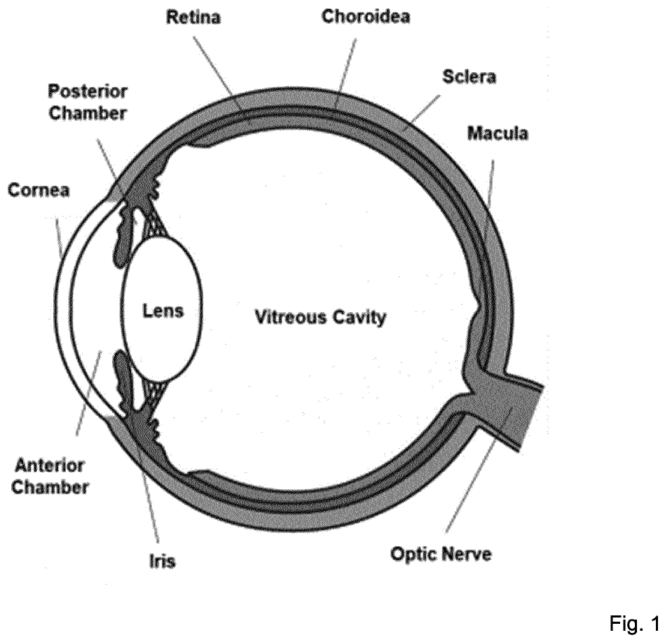

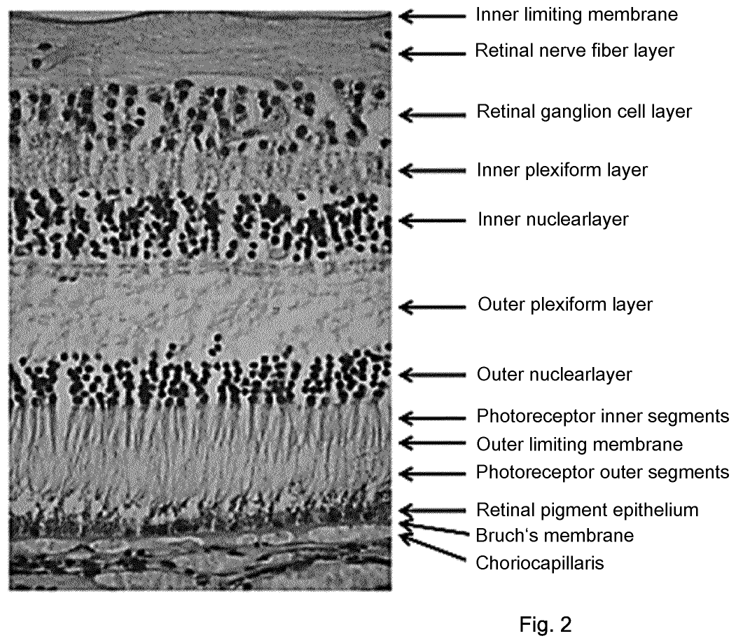

### DETAILED DESCRIPTION OF THE INVENTION

The present invention relates to an EGFR agonist for use in the treatment of an RPE damage-associated disease in a patient.

The RPE damage-associated disease is preferably selected from AMD, retinitis pigmentosa, cone-rod dystrophies or cone dystrophies, polypoidal choroidal vasculopathy, and Stargardt's disease. In particular, the invention relates to pharmaceutical compositions comprising the EGFR agonist.

As hitherto unpublished, and as shown in the examples, the intravitreal application of EGF into the eye of patients with geographic atrophy as the late stage of AMD resulted in an improvement in visual function and macular morphology, including an increase in visual acuity, an increase in the perimetric light differential threshold in the visual center, an improvement in metamorphopsias, and a slight increase in the thickness of the macular tissue (see Examples 1 and 2). Subjectively, the patients noted an improvement in vision with higher clarity and less visual distortion.

Moreover, EGF was well tolerated intraocularly without signs of intraocular inflammation or toxicity. The non-toxicity was first tested in both rabbits and guinea pigs (see Examples 3 and 4).

The molecular mechanism for the observed effect appears to be the supportive and proliferative signal of the EGFR activation upon administration of the EGF. This conclusion is derivable from the enhancement of RPE culture proliferation and migration in vitro upon contacting of the RPE culture with EGF (see Example 5). It was shown that the addition of EGF caused an increase in EGFR signaling via the PI3K/Akt pathway leading ultimately to the increased proliferation and migration of the RPE cells.

This experiment also confirms earlier studies, in which the effect of EGF in RPE cell culture has been tested. Considering the early establishment of RPE cell lines, a myriad of studies have been carried out with RPE cell lines, including studies testing the effect of growth factors, such as platelet derived growth factor (PDGF) or EGF on RPE cell lines.

For example, Spraul and colleagues examined in 2004 the effects of platelet-derived growth factor (PDGF), basic fibroblast growth factor (bFGF), acidic fibroblast growth factor (aFGF), insulin-like growth factor-1 (IGF-1), EGF, and transforming growth factor beta 2 (TGF(beta2)) on bovine RPE cell migration and proliferation in vitro. They found that the RPE cell migration was significantly enhanced after incubation with PDGF, bFGF, aFGF, IGF-1 and EGF. Moreover, bFGF, PDGF, aFGF, and EGF stimulated RPE cell DNA synthesis (see Spraul et al. 2004).

Around the same time, Defoe and colleagues showed that EGF-stimulated survival of RPE D407 cells takes place as a result of signaling through both PI3K and ERK/MAPK pathways, and that residual anti-apoptotic activity stimulated by EGF in the presence of both blockers suggests that additional as yet unidentified growth factor-dependent survival pathways exist (see Defoe et al. 2004). Moreover, it has been described in 2007 that EGF induces the EGF-EGFR-MAPK (mitogen-activated protein kinase) signal transduction pathway in human RPE cells in culture in a concentration-dependent manner. The authors speculated that this may play a role in the activation of human RPE cell proliferation (see Yan et al. 2007).

Despite the knowledge about the effect of growth factors on the proliferation of RPE cell lines, growth factors have never been used in patient treatment, not even clinical trials for an RPE associated disease. Instead, growth factors including bFGF and EGF have been included in the recipes of cell culture media (see Fronk and Vargis, 2016).

In contrast, the present inventors are the first to identify that EGF has a clinically relevant effect on the RPE and, therefore, on the condition of the treated AMD patients. Moreover, the studies confirmed that the effect involves the activation of EGFR. It is hereby postulated that due to its ectodermal lineage, the EGFR activation support the RPE in general, and in particular in the macular region as the region with the highest exposure of the RPE to internal stimuli (such as the photoreceptor outer segment renewal and photo-pigment recycling) and external stimuli such as light and heat.

The notion of EGF being helpful for the treatment of RPE-related diseases is further supported by the finding that the RPE expresses the EGFR (Yan et al. 2007).

According to the invention, EGFR agonists include direct and indirect EGFR agonists. A direct EGFR agonist is any protein or other molecule that, like EGF has the capability of binding to and activating the EGFR leading to an increase in the activity of the downstream signaling pathways of EGFR. An indirect EGFR agonist is any protein or other molecule that induces the expression of an endogenous protein that has the capability of binding to and activating the EGFR leading to an increase in the activity of the downstream signaling pathways of EGFR.

The EGFR (also referred to as ErbB-1 or HER1 in humans) is a transmembrane protein and tyrosine kinase that is a receptor for and activated by members of the epidermal growth factor family (EGF family) of extracellular protein ligands (Herbst et al. 2004). It is noted that the EGFR belongs to the group of ErbB receptors (ErbBs), which consists of four transmembrane receptors belonging to the receptor tyrosine kinase (RTK) superfamily and includes EGFR (ErbB1/HER1), ErbB2/Neu/HER2, ErbB3/HER3, and ErbB4/HER4 (see Wikipedia article on EGFR). All four ErbBs have a common structure, with an extracellular ligand-binding domain, a single membrane-spanning region, a homologic cytoplasmic protein tyrosine kinase domain and a C-terminal tail with multiple phosphorylation sites.

The engagement of the receptor by EGF induces a series of intracellular mitogenic signal transduction pathways, consequently leading to processes such as cell proliferation, migration and differentiation.

Members of the EGF family have highly similar structural and functional characteristics. The ability of the EGF-family members to bind EGFR is based on the common presence of one or more repeats of the “EGFR binding motif” with the conserved amino acid sequence:

(SEQ ID NO: 1)
CX₇CX₄₋₅CX₁₀₋₁₃CXCX₅GXRC

with X representing any amino acid.

This sequence contains six cysteine residues that form three intramolecular disulfide bonds. Disulfide bond formation generates three structural loops that are essential for high-affinity binding between members of the EGF-family and their cell-surface receptors.

According to one embodiment of the invention, the EGFR agonist is a protein comprising the EGF family consensus amino acid sequence: CX₇CX₄₋₅CX₁₀₋₁₃CXCX₅GXRC (SEQ ID NO: 1), wherein X is any proteogenic amino acid. With this sequence, it is guaranteed that the EGFR agonist is able to “increase the epidermal growth factor receptor (EGFR) signaling”.

As used herein the ability to “increase the EGFR signaling” indicates the capability of the agents of the present invention to increase the activity of the downstream signaling pathways of the respective receptors.

Besides EGF itself other family members comprise: Heparin-binding EGF-like growth factor (HB-EGF), transforming growth factor-α (TGF-α), amphiregulin (AR), epiregulin (EPR), epigen, Betacellulin (BTC), neuregulin-1 (NRG1), neuregulin-2 (NRG2), neuregulin-3 (NRG3), neuregulin-4 (NRG4).

EGF family members are best known for their ability to stimulate cell growth and proliferation and are important for many developmental processes including promoting mitogenesis and differentiation of mesenchymal and epithelial cells. Therefore, it is reasonable to expect that, in addition to EGF, which by binding to EGFR excerpts an effect of the proliferation and migration of RPE cells upon contact, also the other members of the family have the same effect.

According to one embodiment, the EGFR agonist comprises an amino acid sequence with an identity to a member of the EGF family a fragment thereof of at least 80%, at least 90%, at least 95%, at least 98% or 100%. The relatedness between two amino acid sequences or between two nucleotide sequences is described by the parameter “sequence identity”. For purposes of the present invention, the degree of sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 3.0.0 or later. The optional parameters used are gap open penalty of 10, gap extension penalty of 0.5, and the EBLOSUM62 (EMBOSS version of BLOSUM62) substitution matrix. The output of Needle labeled “longest identity” is used as the percent identity and is calculated as follows:

(Identical Residues×100)/(Length of Alignment−Total Number of Gaps in Alignment)

According to one embodiment, the member of the EGF family is selected from EGF, HB-EGF, TGF-α, AR, EPR, epigen, BTC, NRG1, NRG2, NRG3 and NRG4.

The EGF family members can be divided into three groups based on their affinity to ErbB. The first group including EGF, AR, TGF-α bind only to EGFR. The second group including BTC, HB-EGF, and EPR exhibits dual specificity in that they bind both EGFR and ErbB4. The third group is composed of the neuregulins and forms two subgroups based upon their capacity to bind ErbB3 and ErbB4 (NRG-1 and NRG-2) or only ErbB4 (NRG-3 and NRG-4). Thus, according to one embodiment, the member of the EGF family is selected from EGF, AR, and TGF-α.

All members of the EGF family are synthetized in vivo as pre-pro-proteins, i.e. including an N-terminal signal peptide and a pro-sequence. The N-terminal signal peptide directs the pro-protein to the membrane (being cleaved off in the endoplasmic reticulum). For activation, the mature proteins are cleaved of membrane bound pro-sequence.

The full sequence of pre-pro-EGF is shown in UniprotKB P01133. The mature human EGF is a 6-kDa protein with 53 amino acid residues with the SEQ ID NO: 2. According to one embodiment the indirect EGFR agonist induces the expression of endogenous EGF.

The EGFR binding motif of EGF is CPLSHDGYCLHDGVCMYIEALDK-YACNCVVGYIGERC (SEQ ID NO: 3). Importantly, the EGFR binding motif makes up 36 out of the total 53 amino acids of EGF (67%). As the EGFR binding motif is not only necessary but sufficient for binding to EGFR and the EGF only contains 17 additional amino acids, which most likely serve structural purposes, it is reasonable to conclude that any protein with the EGFR binding motif can bind to EGFR and activate signaling cascade.

Thus, according to one embodiment, the EGFR agonist comprises an amino acid sequence with an identity of at least 80%, at least 90%, at least 95%, at least 98% or 100% to SEQ ID NO: 2 (EGF) or a fragment thereof. In particular, the amino acid sequence includes the EGFR binding motif of EGF, namely

(SEQ ID NO: 3)
CPLSHDGYCLHDGVCMYIEALDKYACNCWGYIGERC

According to one embodiment, the EGFR agonist comprises SEQ ID NO: 2 or a fragment thereof. According to one embodiment, the EGFR agonist consists of SEQ ID NO: 1, Pro-EGF according to UniprotKB P30111 or a fragment thereof.

In this context, the term “fragment” as used herein relates to fragments of the reference protein that have one or more amino acid deletions with respect to full sequence. As the fragment must contain the EGFR binding motif, the number of deletions is limited and it is guaranteed that the fragment is able to bind and activate the EGFR.

According to one embodiment the indirect EGFR agonist induces the expression of the endogenous EGF gene.

Amphiregulin (AR) is a protein synthetized as a transmembrane glycoprotein with 252 amino acids. For activation, the mature AR consisting of 87 amino acid is cleaved of the pro-sequence. AR is encoded by the AREG gene in human. According to one embodiment the indirect EGFR agonist induces the expression of the endogenous AREG gene. The expression of the AREG gene is induced through the activation of the cAMP/PKA pathway by prostaglandin, the protein kinase C pathway, parathyroid hormone, polycystin-1. AREG expression is also induced by cytokines such as interleukin-1, and tumor necrosis factor, or hormones such as androgens, parathyroid hormone, insulin, and estrogens (see Berasain et al. 2014). Accordingly, suitable indirect EGFR agonists according to the invention are prostaglandins, parathyroid hormone, polycystin-1, interleukin-1, tumor necrosis factor, androgens, parathyroid hormone, insulin, and estrogens. The sequence of the mature amphiregulin is identified herein as SEQ ID NO: 4.

Thus, according to one embodiment, the EGFR agonist comprises an amino acid sequence with an identity of at least 80%, at least 90%, at least 95%, at least 98% or 100% to SEQ ID NO: 4 (AR) or a fragment thereof. In particular, the amino acid sequence includes the EGFR binding motif of AR, namely CNAEFQNFCIHGECK YIEHLEAVTCKCQQEYFGERC (SEQ ID NO: 5).

According to one embodiment, the EGFR agonist comprises SEQ ID NO: 4 (AR) or a fragment thereof. According to one embodiment, the EGFR agonist consists of pro-amphiregulin, amphiregulin or a fragment thereof.

Heparin-binding EGF-like growth factor (HB-EGF) is synthetized as a transmembrane protein with 208 amino acids. According to one embodiment the indirect EGFR agonist induces the expression of the endogenous HB-EGF gene. For activation, the mature HB-EGF consisting of 85 amino acid is cleaved of the pro-sequence. The sequence of the mature HB-EGF is identified herein as SEQ ID NO: 6.

Thus, according to one embodiment, the EGFR agonist comprises an amino acid sequence with an identity of at least 80%, at least 90%, at least 95%, at least 98% or 100% to SEQ ID NO: 6 (HB-EGF) or a fragment thereof. In particular, the amino acid sequence includes the EGFR binding motif of HB-EGF, namely CLRKYKDFCIHGE CKYVKELRAPSCICHPGYHGERC (SEQ ID NO: 7).

According to one embodiment, the EGFR agonist comprises SEQ ID NO: 6 (HB-EGF) or a fragment thereof. According to one embodiment, the EGFR agonist consists of pro-HB-EGF, HB-EGF or a fragment thereof.

Transforming growth factor-α (TGF-α) is synthetized as a transmembrane protein with 160 amino acids. According to one embodiment the indirect EGFR agonist induces the expression of the endogenous TGF-α gene. For activation, the mature TGF-α consisting of 50 amino acid is cleaved of the pro-sequence. The sequence of the mature TGF-α is identified herein as SEQ ID NO: 8.

Thus, according to one embodiment, the EGFR agonist comprises an amino acid sequence with an identity of at least 80%, at least 90%, at least 95%, at least 98% or 100% to SEQ ID NO: 8 (TGF-α) or a fragment thereof. In particular, the amino acid sequence includes the EGFR binding motif of TGF-α, namely

(SEQ ID NO: 9)
CPDSHTQFCFHGTCRFLVQEDKPACVCHSGYVGARC

According to one embodiment, the EGFR agonist comprises SEQ ID NO: 8 (TGF-α) or a fragment thereof. According to one embodiment, the EGFR agonist consists of pro-TGF-α, TGF-α or a fragment thereof.

Epiregulin (EPR) is synthetized as a transmembrane protein with 154 amino acids. According to one embodiment the indirect EGFR agonist induces the expression of the endogenous EPR gene. The mature membrane bound EPR after removal of the signal peptide consists of 132 amino acids. The sequence of the mature EPR is identified herein as SEQ ID NO: 10.

Thus, according to one embodiment, the EGFR agonist comprises an amino acid sequence with an identity of at least 80%, at least 90%, at least 95%, at least 98% or 100% to SEQ ID NO: 10 (EPR) or a fragment thereof. In particular, the amino acid sequence includes the EGFR binding motif of EPR, namely CLEDHNSYCIN GACAFHHELE KAICRCFTGYTGERC (SEQ ID NO: 11).

According to one embodiment, the EGFR agonist comprises SEQ ID NO: 10 (EPR) or a fragment thereof. According to one embodiment, the EGFR agonist preferably consists of pro-EPR, EPR or a fragment thereof.

Betacellulin (BTC) is synthetized as a transmembrane protein with 178 amino acids. According to one embodiment the indirect EGFR agonist induces the expression of the endogenous BTC gene. For activation, the mature BTC consisting of 80 amino acid is cleaved off of the pro-sequence. The sequence of the mature BTC is identified herein as SEQ ID NO: 12.

Thus, according to one embodiment, the EGFR agonist comprises an amino acid sequence with an identity to of at least 80%, at least 90%, at least 95%, at least 98% or 100% to SEQ ID NO: 12 (BTC) or a fragment thereof. In particular, the amino acid sequence includes the EGFR binding motif of BTC, namely CPKQYKHYCIKG RCRFVVAEQTPSCVCDEGYIGARC (SEQ ID NO: 13).

According to one embodiment, the EGFR agonist comprises SEQ ID NO: 12 (BTC) or a fragment thereof. According to one embodiment, the EGFR agonist consists of pro-BTC, BTC or a fragment thereof.

Neuregulin-1 (NRG-1) is synthetized as a transmembrane protein with 640 amino acids. According to one embodiment, the indirect EGFR agonist induces the expression of the endogenous NRG-1 gene. For activation, the mature NRG-1 consisting of 222 amino acid is cleaved of the pro-sequence. The sequence of the mature NRG-1 is identified herein as SEQ ID NO: 14.

Thus, according to one embodiment, the EGFR agonist comprises an amino acid sequence with an identity to SEQ ID NO: 14 (NRG-1) or a fragment of at least 80%, at least 90%, at least 95%, at least 98% or 100%. In particular, the amino acid sequence includes the EGFR binding motif of NRG-1, namely CAEKEKTFC VNGGECFMVKDLSNPSRYLCKCQPGFTGARC (SEQ ID NO: 15).

According to one embodiment, the EGFR agonist comprises SEQ ID NO: 14 (NRG-1) or a fragment thereof. According to one embodiment, the EGFR agonist consists of pro-NRG-1, NRG-1 or a fragment thereof.

Neuregulin-2 (NRG-2) is synthetized as a transmembrane protein with 850 amino acids. According to one embodiment, the indirect EGFR agonist induces the expression of the endogenous NRG-2 gene. For activation, the mature NRG-2 consisting of 293 amino acid is cleaved of the pro-sequence. The sequence of the mature NRG-2 is identified herein as SEQ ID NO: 16.

Thus, according to one embodiment, the EGFR agonist comprises an amino acid sequence with an identity to of at least 80%, at least 90%, at least 95%, at least 98% or 100% to SEQ ID NO: 16 (NRG-2) or a fragment thereof. In particular, the amino acid sequence includes the EGFR binding motif of NRG-2, namely CNETAK SYCVNGGVCYYIEGINQLSCKCPNGFFGQRC (SEQ ID NO: 17).

According to one embodiment, the EGFR agonist comprises SEQ ID NO: 16 (NRG 2) or a fragment thereof. According to one embodiment, the EGFR agonist preferably consists of pro-NRG-2, NRG-2 or a fragment thereof.

Neuregulin-3 (NRG-3) is synthetized as a transmembrane protein with 720 amino acids. According to one embodiment, the indirect EGFR agonist induces the expression of the endogenous NRG-3 gene. The extracellular part of the mature protein consists of 359 amino acid. The sequence of the mature NRG-3 is identified herein as SEQ ID NO: 18.

Thus, according to one embodiment, the EGFR agonist comprises an amino acid sequence with an identity of at least 80%, at least 90%, at least 95%, at least 98% or 100% to SEQ ID NO: 18 (NRG-3) or a fragment thereof. In particular, the amino acid sequence includes the EGFR binding motif of NRG-3, namely

(SEQ ID NO: 19)
CRDKDLAYCLNDGECFVIETLTGSHKHCRCKEGYQGVRC

According to one embodiment, the EGFR agonist comprises SEQ ID NO: 16 (NRG 2) or a fragment thereof. According to one embodiment, the EGFR agonist preferably consists of pro-NRG-3, NRG-3 or a fragment thereof.

Neuregulin-4 (NRG-4) is synthetized as a transmembrane protein with 114 amino acids. According to one embodiment, the indirect EGFR agonist induces the expression of the endogenous NRG-4 gene. For activation, the mature NRG-4 consisting of 61 amino acid is cleaved of the pro-sequence. The sequence of the mature NRG-4 is identified herein as SEQ ID NO: 20.

Thus, according to one embodiment, the EGFR agonist comprises an amino acid sequence with an identity of at least 80%, at least 90%, at least 95%, at least 98% or 100% to SEQ ID NO: 20 (NRG-4) or a fragment thereof. In particular, the amino acid sequence includes the EGFR binding motif of NRG-4, namely CG PSHKSFCLNG GLCYVIPTIP SPFCRCVENYTGARC (SEQ ID NO: 21).

According to one embodiment, the EGFR agonist comprises SEQ ID NO: 20 (NRG-4) or a fragment thereof. According to one embodiment, the EGFR agonist preferably consists of pro-NRG-4, NRG-4 or a fragment thereof.

The EGFR agonist may be a native protein isolated from an organism or a recombinantly produced protein. According to one embodiment, the EGFR agonist is recombinantly expressed in bacteria, such as E. coli, or eukaryotic cells, such as a mammalian, insect, plant, or fungal cell.

The EGFR agonist may be a naturally occurring EGF member or a recombinant fusion protein comprising a EGFR binding sequence of an EGF member. The term “fusion protein” according to the invention relates to proteins created through the joining of two or more genes, cDNAs or sequences that originally coded for separate proteins/peptides. The genes may be naturally occurring in the same organism or different organisms or may synthetic polynucleotides. In addition, an EGFR agonist fusion protein may additionally comprise functional fusion peptides. The fusion peptides may have the function of increasing the stability and/or half-life of the EGFR agonist. Suitable fusion peptides are for example antibody fragments in particular Fc-fragments. Other suitable fusion peptides are binding sites for posttranslational modifications such as hydroxyethyl starch (HES) or polyethylene glycol (PEG) or glycosylation.

A “peptide” as used herein may be composed of any number of amino acids of any type, preferably naturally occurring amino acids, which, preferably, are linked by peptide bonds. In particular, a peptide comprises at least 3 amino acids, preferably at least 5, at least 7, at least 9, at least 12, or at least 15 amino acids. Furthermore, there is no upper limit for the length of a peptide. The term “protein” refers to a peptide with at least 40, at least 60, at least 80, preferably at least 100 amino acids.

The EGFR agonist is in particular an isolated protein. Methods for the isolation and/or purification of naturally EGF family members or artificial EGFR agonists are not particularly limited and are known in the art. Further, methods for the generation and expression of recombinant EGF family members and EGF family member containing proteins are not particularly limited and are known in the art.

The patient to be treated can be any mammal, including, but not limited to, mammals of the order Rodentia, such as mice and hamsters, and mammals of the order Logomorpha, such as rabbits, from the order Carnivora, including Felines (cats) and Canines (dogs) of the order Artiodactyla, including Bovines (cows) and Swines (pigs) or of the order Perssodactyla, including Equines (horses), of the order Primates, Ceboids, or Simoids (monkeys) or of the order Anthropoids (humans and apes). An especially preferred mammal is the human. According to a preferred embodiment, the patient is human.

As the EGFR agonists according to the invention have a positive, direct or indirect, effect on the growth of the RPE cells and, consequently, as shown in the examples have a positive effect on AMD, it is concluded that also other RPE damage associated diseases such as retinitis pigmentosa, cone-rod dystrophies or cone dystrophies, polypoidal choroidal vasculopathy, or Stargardt's disease, can be treated by the EGFR agonists according to the invention. The basis for this statement is, that as described above, the RPE in all these diseases is primarily or secondarily affected, so that any support for the RPE by an activation of the EGFR and/or by a direct or indirect enhancement of the effect of an EGFR activation, will have a positive effect on the course of the diseases. According to one embodiment, the RPE damage associated disease is AMD.

In a preferred embodiment, the pharmaceutical composition does not comprise stem cells for treating the RPE damage.

The pharmaceutical composition comprising the EGFR agonist may be administered intravitreally, epicorneally, transcorneally, transsclerally, transconjunctivally, subconjunctivally, intraocularly or into the Tenon's space.

Needles for administering the EGFR agonist are known in the art. Possible needle sizes according to the Birmingham wire gauge system are gauge 25, 26, 27, 28, 29, 30, 31, 32, 33, 34. According to one embodiment, the needle size is selected from gauge 28, 29, 30, 31, 32. According to one embodiment, the needle size is gauge 30. Syringes for administering the EGFR agonist are known in the art. Suitable syringe sizes are 0.5 ml, 1.0 ml, 2.5 ml or 5 ml. In particular, tuberculin syringes (volume 1 mL), are well suited.

Preferably, the pharmaceutical composition is administered intravitreally. For the intravitreal application, it is suggested to apply anesthesia. In particular, anesthesia of the cornea and conjunctiva is obtained by applying topical anesthetic eye drops (such as oxybuprocain). The external ocular surface as well as the lid margins and the lids are disinfected and a lid speculum is inserted.

The pharmaceutical composition may be injected at different locations of the eye. According to one embodiment, the pharmaceutical composition is injected in the temporal inferior quadrant. According to one embodiment, the pharmaceutical composition is injected posterior to the corneal limbus. According to one embodiment, the pharmaceutical composition is injected through the conjunctiva, sclera and pars plana into the vitreous cavity.

According to one embodiment, the pharmaceutical composition is injected in a distance of 1 mm to 6 mm posterior to the corneal limbus. According to one embodiment, the pharmaceutical composition is injected in a distance of 2 mm to 5 mm posterior to the corneal limbus. According to one embodiment, the pharmaceutical composition is injected in a distance of 3 mm to 4 mm posterior to the corneal limbus.

After the needle is withdrawn an ointment containing an antibiotic and an anti-inflammatory agent may be applied.

According to one embodiment, the EGFR agonist is administered in the pharmaceutical composition in a general dosage in the range of 0.30 μg to 600 μg per eye. Accordingly, the EGFR agonist is administered, for example, in a dosage of 0.30 μg, 0.35 μg, 0.40 μg, 0.45 μg 0.50 μg, 0.75 μg, 1.5 μg, 5 μg, 10 μg, 20 μg, 30 μg, 40 μg, 50 μg, 55 μg, 60 μg, 65 μg, 70 μg, 75 μg, 80 μg, 85 μg, 90 μg, 95 μg, 100 μg. 120 μg, 140 μg, 160 μg, 180 μg, 200 μg, 220 μg, 240 μg, 260 μg, 280 μg, 300 μg, 320 μg, 340 μg, 360 μg, 380 μg, 400 μg, 420 μg, 440 μg, 460 μg, 480 μg, 500 μg, 520 μg, 540 μg, 560 μg, 580 μg, or 600 μg.

In a dosage of less than 0.30 μg, no significant therapeutic or prophylactic effect is measured. A dosage above 600 μg does not further improve the pharmaceutical effect and increases the risk of side effects. According to one embodiment, the EGFR agonist is administered in the pharmaceutical composition in a dosage in the range of 0.30 μg to 200 μg. According to one embodiment, the EGFR agonist is administered in the pharmaceutical composition in a dosage in the range of 0.50 μg to 100 μg. The relatively wide range of the applicable dose is due to the fact that EGF is a physiological growth factor, so that in contrast to non-physiological molecules it has a relatively wide range of tolerability and efficacy.

Moreover, it is expected that there is a high dosage optimum and a low dosage optimum. Considering the low dosage optimum, the EGFR agonist may be administered in the range of 0.50 μg to 10 μg. According to one embodiment, the EGFR agonist is administered in the pharmaceutical composition in a dosage in the range of 0.50 μg to 1.0 μg, preferably in a dosage in the range of 0.60 μg to 0.90 μg For example, the EGFR agonist is administered in a dosage of 0.50 μg, 0.55 μg, 0.60 μg, 0.65 μg, 0.70 μg, 0.75 μg, 0.80 μg, 0.85 μg, 0.90 μg, 0.95 μg, 1.0 μg.

Considering the high dosage optimum, the EGFR agonist may be administered in a dosage in the range of 50 μg to 100 μg, preferably in a in a dosage in the range of 60 μg to 90 μg.

The administration of the EGFR agonist is expected to have low side effects. As shown in Examples 3 and 4, neither in the rabbits nor in the guinea pigs, any injection-related effects such as a loss of retinal cells, a change in the intravitally measured optical coherence tomography (OCT) based retinal thickness measurements, an increase in the number of apoptotic retinal cells, a shrinkage or swelling of the ciliary body, and induction of astrogliosis, or changes in the intraocular pressure were noted. In the same manner, there were no signs of intraocular inflammation detected, neither during the intravital examination nor upon histological examinations of the globes. The findings support the notion, that repeatedly intravitreally applied EGF and amphiregulin did not result in an intraocular inflammatory or toxic effect. It supports the notion of safety of intraocular applications of the EGF and amphiregulin.

The pharmaceutical composition may be in any suitable dosage form for administration to the patient, for example in form of crystals, a solution or a lyophilisate. According to one embodiment, the pharmaceutical composition is a solution or a lyophilisate.

In a preferred embodiment, the EGFR agonist or a pharmaceutically acceptable salt thereof is formulated in the pharmaceutical composition with one or more pharmaceutically acceptable excipient(s) and/or carrier(s).

The lyophilized protein may be reconstituted in sterile buffer. Suitable buffers components are, for example citrate or sodium phosphate. Sodium phosphate buffer consists of sodium dihydrogen phosphate (NaH₂PO₄) and sodium dihydrogen phosphate (Na₂HPO₄).

In order to increase the stability of the EGF in solution allowing a longer storage time, the buffered solution may be supplemented with stabilizers. Suitable stabilizers include sucrose, dextran, and carrier proteins such as heat inactivated fetal calf serum (FCS) or tissue culture grade bovine serum albumin (BSA). According to one embodiment the lyophilized EGFR agonist is reconstituted in water for injection. Accordingly, in the pharmaceutical composition, the EGFR agonist may be solubilized in water for injection.

According to an alternative embodiment, the EGFR agonist is formulated with sucrose, dextran and a sodium phosphate buffer.

The concentration of the EGFR agonist in the pharmaceutical composition may be in the range of 0.006 g/l to 12 g/l. For example, the concentration of the EGFR agonist in the pharmaceutical composition may be 0.006 g/l, 0.01 g/l, 0.015 g/l, 0.02 g/l, 0.03 g/l, 0.04 g/l, 0.05 g/l, 0,06 g/l, 0.07 g/l, 0,08 g/l, 0.09 g/l, 0.1 g/l, 0.2 g/l, 0.3 g/l, 0.4 g/l, 0.5 g/l, 0.6 g/l, 0.7 g/l, 0.8 g/l, 0.9 g/l, 1.0 g/l, 1.1 g/l, 1.2 g/l, 1.3 g/l, 1.4 g/l, 1.5 g/l, 1.6 g/l, 1.7 g/l, 1.8 g/l, 1.9 g/l, 2.0 g/l, 2.5 g/l, 3.0 g/l, 3.5 g/l, 4.0 g/l, 4.5 g/l, 5.0 g/l, 5.5 g/l, 6.0 g/l, 6.5 g/l, 7.0 g/l, 7.5 g/l, 8.0 g/l, 8.5 g/l, 9.0 g/l, 9.5 g/l, 10.0 g/l, 10.5 g/l, 11.0 g/l, 11.5 g/l, or 12.0 g/l. According to one embodiment, the concentration of the EGFR agonist is in the range of 0.01 g/l up to 8 g/l. According to one embodiment, the concentration of the EGFR agonist is in the range of 0.1 g/l up to 6 g/l.

Considering the low dosage optimum, the concentration of the EGFR agonist may be in the range of 0.01 g/l up to 0.2 g/l. According to one embodiment, the concentration of the EGFR agonist is in the range of 0.01 g/l up to 0.02 g/l. Considering the high dosage optimum, the concentration of the EGFR agonist may be in the range of 1 g/l up to 2 g/l.

The volume to be injected may range from 30 μL up to 200 μL. For example, the volume may be 30 μL, 40 μL, 50 μL, 60 μL, 70 μL, 80 μL, 90 μL, 100 μL, 120 μL, 140 μL, 160 μL, 180 μL, or 200 μL. According to one embodiment, the volume is in the range of 50 μL up to 100 μL. For volumes of more 100 μL, a paracentesis would be performed to release fluid from the anterior chamber and to reduce the ocular volume before the injection.

The treatment may be a one-time treatment. Preferably, the pharmaceutical composition is administered multiple times. The time interval between the administrations may be in the range of 2 days to 6 months. For example, The time interval between the administrations is 2 days, 3 days, 4 days, 5 days, 6 days, 7 days, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months. According to one embodiment, the time interval between the administrations is in the range of 2 weeks to 8 weeks. According to one embodiment, the time interval between the administrations is in the range of 3 weeks to 5 weeks.

The administration of the EGFR agonist at least impairs the progress of the loss of visual acuity, in particular in the case of an AMD patient. Thus, according to one embodiment the administration of the EGFR agonist maintains the visual acuity at a constant level (±5 ETDRS letter). The visual acuity may be maintained at a constant level of ±5 ETDRS letters over a period of at least 12 months. According to one embodiment, the visual acuity may be maintained at a constant level of ±5 EDTRS letters over a period of at least 18 months. According to one embodiment, the visual acuity may be maintained at a constant level ±5 ETDRS letters over a period of at least 24 months. In contrast, visual acuity decreased in untreated patients in clinical studies, such as the PROXIMA A study, at 24 months after baseline by 13.88±1.40 ETDRS letters (Holekamp et al. 2019).

Preferably, the visual acuity is not only stabilized but even improved. According to one embodiment, the visual acuity is improved by the treatment by at least 2 ETDRS letters, at least 3 ETDRS letters, at least 4 ETDRS letters, at least 5 ETDRS letters, at least 6 ETDRS letters, at least 7 ETDRS letters, at least 8 ETDRS letters, at least 9 ETDRS letters, at least 10 ETDRS letters.

The injections may be performed on an out-patient basis. In dependence of the clinical situation, check-up examinations are preferably carried out at the first day after the injection and later in regular intervals.

### EXAMPLES

### Example 1—Results of the First Patient with Geographic Atrophy and Treated with Intravitreally Applied EGF

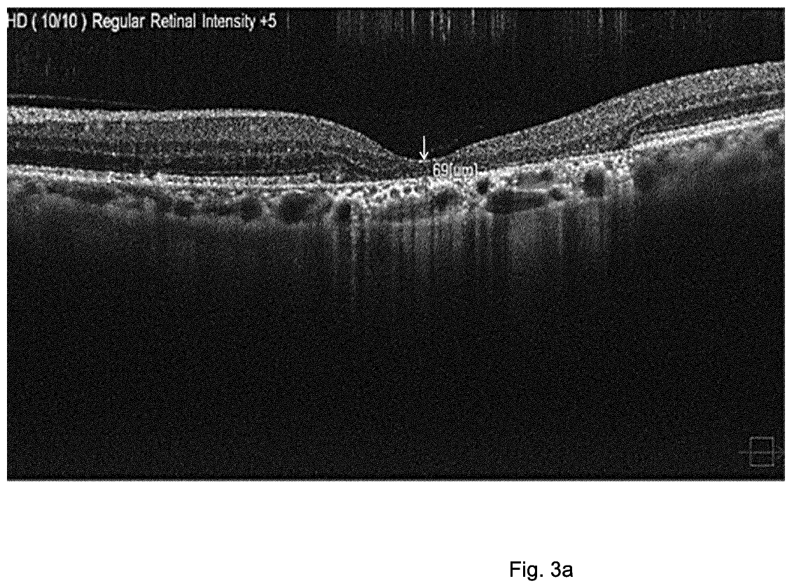

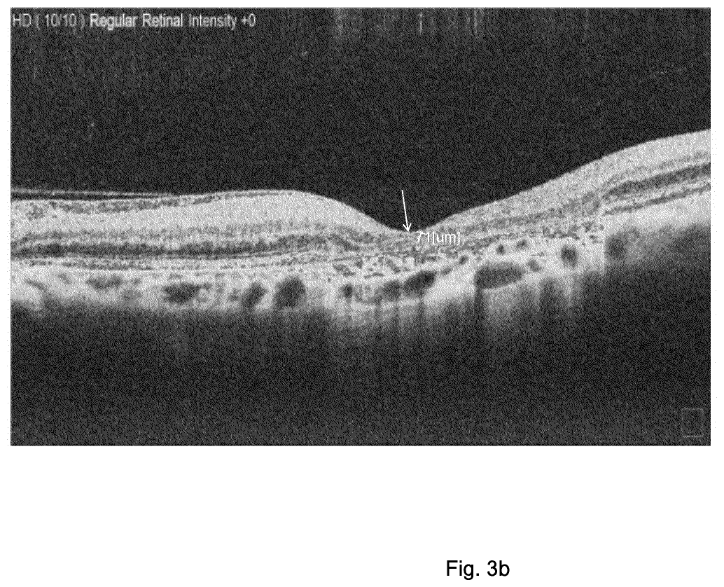

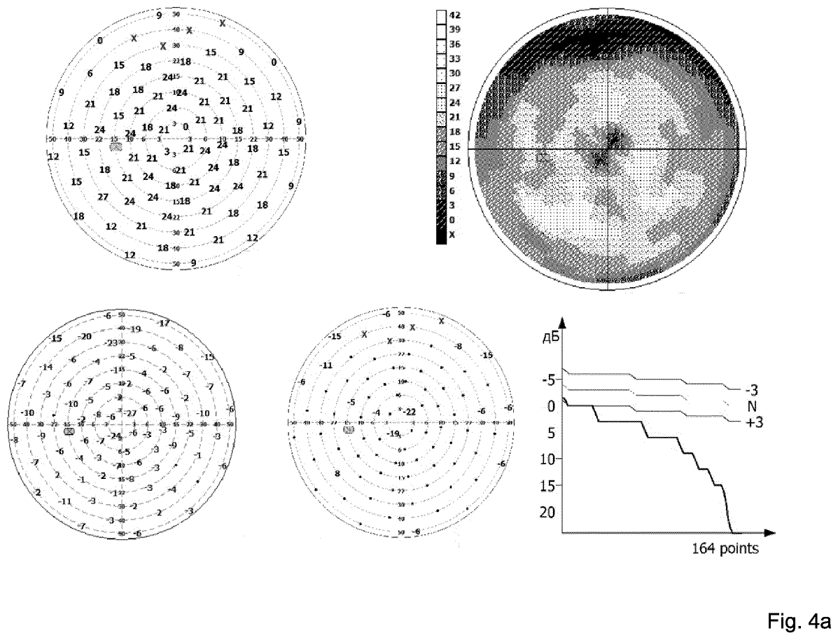

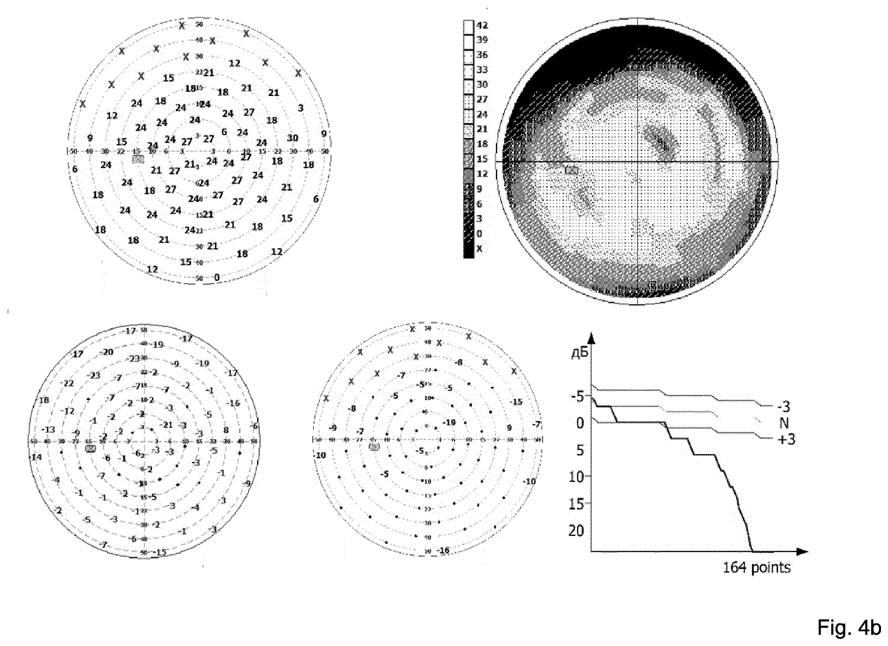

A 68-years old patient U. with geographic atrophy was treated with EGF;
On the first day after injection, there were no pathological reactions of the anterior segment of the eye. The intraocular pressure was unchanged within normal limits. Subjectively, the patient did not notice any changes.
On the third day, a mixed moderate conjunctival injection appeared on the surface of the eye, without any other changes in the anterior or posterior segment of the eye. Subjectively, the patient felt an improvement in vision: the distortions and curvatures of objects (metamorphopsias) disappeared. The peripheral vision remained unchanged; the central visual acuity was unchanged. Optical coherence tomography images of the fundus signs were unchanged.
On the 7th day after the injection, the eye appeared to be untouched, the conjunctival injection had subsided. Visual acuity had improved from 0.03 before the injection to 0.1. Also subjectively, the patient noted a marked improvement in vision with an increase in the clarity, and the objects no longer appeared to be distorted, and the vertical lines were no longer curved. Upon optical coherence tomography, the foveolar zone increased in thickness from 69 μm at baseline to 71 μm at seven days after the injection, and to 73 μm two weeks later (FIGS. 3a, b). Computer perimetry revealed an increase in the light differential sensitivity in the central zone from 11.3 dB per measurement point (taking the central four measurement points) to 24.8 dB per measurement point (FIGS. 4a, b).

### Example 2—Treatment of AMD Patients with EGF

A series of seven patients were consecutively treated. At baseline, all patients received an intravitreal injection of 0.75 μg EGF in 50 μl, corresponding to a concentration of 0.015 μg EGF/μl. The patients underwent weekly to two-weekly ophthalmological follow-up examinations. Three patients (patients #1, 2, and 3) received a repeated intravitreal injection after an interval of three to four weeks.

Patients with Medical Re-Examinations:

| | | | Visual | Visual | | | |
|---|---|---|---|---|---|---|---|
| | | | Acuity | Acuity | | | |
| | | | (Decimal) | (Decimal) | Improvement | | Intraocular |
| | | Date of | before | at last | in ETDRS | Quality | Pressure |
| Patient | Eye | injection | Injection | examination | letters | of vision | (mmHg)P |
| 1 | OS | Sep. 12, 2019 | 0.03 | 0.1 | 26 | Improved | 12 |
| 2 | OD | Apr. 2, 2020 | 0.06 | 0.08 | 6 | Improved | 14 |
| 3 | OS | Apr. 2, 2020 | 0.03 | 0.05 | 11 | Improved | 16 |
| 4 | OS | Nov. 2, 2020 | 0.1 | 0.1 | 0 | Improved | 19 |
| 5 | OD | Nov. 2, 2020 | 0.08 | 0.09 | 3 | Improved | 17 |
| 6 | OS | Apr. 2, 2020 | 0.02 | 0.03 | 8 | Improved | 13 |
| 7 | OD | Nov. 2, 2020 | 0.04 | 0.06 | 9 | Improved | 18 |

Out of the seven patients, six showed an improvement in their measured visual acuity after a follow-up ranging between one week and seven weeks. Upon fundus examination and upon optical coherence tomography, there was a tendency of an increase in the thickness of the tissue in the foveolar region and changes in the density and amount of macular pigments. Parallel to the improvement in visual acuity, the central visual field also improved as tested by perimetry. Besides a conjunctival vessel dilatation or a circumscribed subconjunctival hemorrhage at the site of the injection, no other side effects were noted. In none of the eyes, cells in the anterior chamber fluid nor in the vitreous cavity were detected upon slit lamp-based biomicroscopy, none of the eyes showed an increase in intraocular pressure beyond a pressure value of 21 mmHg, and none of the eyes revealed hemorrhages or other newly developed pathologies in the retina. There were no signs of intraocular inflammation, lens opacification, changes in the clarity of the optic media, or any significant change in intraocular pressure.

### Example 3—Toxicity Test of Intravitreally Administered EGF (Rabbits)

Purpose: The Purpose of the study was to examine the safety of intravitreally injected epidermal growth factor (EGF).

Methods: The study included four adult rabbits with an age of four months and a body weight of 2.5 kg and which received two intravitreal injections of 100 ng EGF into their right eyes in one-monthly intervals, while their left eyes remained untouched. The injections were performed in the upper right quadrant of the eyes in a distance of 3-4 mm from the limbus. At baseline of the study, and at specific dates after the injection the animals were examined using photography of the external eye and anterior and posterior segment of the eye, tonometry, optical coherence tomography (OCT) of the posterior fundus, and fundus photography. Four weeks after a third injection, the animals were sacrificed and the globes were histomorphometrically examined.

After sacrificing the animals, the eyes were enucleated, immediately fixed in a solution of 1% glutaraldehyde and 4% formaldehyde, and kept in that solution for seven days at room temperature. The sagittal, vertical and horizontal globe diameter were subsequently measured. A central segment with a thickness of about 8 mm and running through the optic nerve head and the pupil was cut out of the fixed globes, dehydrated in alcohol, imbedded in paraffin, sectioned for light microscopy with a slide thickness of 4-6 μm, and stained with hematoxylin eosin.

The cell number in the three retinal layers containing cell nuclei was counted. The slides for apoptotic cell death were additionally stained using the TUNEL (terminal deoxynucleotidyl transferase dUTP nick end labeling) technique.

Results: No significant change in intraocular pressure (IOP) as measured by tonometry and retinal thickness as measured by OCT after the repeated intravitreal injections of EGF (Table 1) were detected. The intravital examinations of the external eye and the anterior segment of the globes did not reveal any sign of toxicity or development of cataract. Upon histomorphometry, differences in the cell count of the various retinal layers, i.e. the retinal photoreceptor cell layer, the outer nuclear layer and the inner nuclear layer, assessed at the posterior pole, the midpoint between the posterior pole and the equator, the equator and in the region posterior to the ora serrata, did not differ significantly (all P-values>0.20) between the eyes injected with EGF and the contralateral eyes injected with PBS. In the TUNEL staining, neither in the right eyes or in the left eyes, apoptotic cells were detected.

TABLE 1
Intraocular pressure and retinal thickness (as measured OCT) in adult rabbits'
eyes at baseline and after intravitreal injections of epidermal growth factor (EGF)

| | | Before | | | | | | | | | | | | | |
|---|---|---|---|---|---|---|---|---|---|---|---|---|---|---|---|
| | | first injection | | 5 Dec. 2018 | | 29 Dec. 2018 | | 13 Jan. 2019 | | 9 Feb. 2019 | | 13 Feb. 2019 | | 26 Mar. 2019 | |
| | | OCT | IOP | OCT | IOP | OCT | IOP | OCT | IOP | OCT | IOP | OCT | IOP | OCT | IOP |
| | Eye | [nm] | [mmHg] | [nm] | [mmHg] | [nm] | [mmHg] | [nm] | [mmHg] | [nm] | [mmHg] | [nm] | [mmHg] | [nm] | [mmHg] |
| 1 | OD | 152 | 7 | 156 | 6 | 160 | 8 | 153 | 11 | 154 | 5 | 152 | 6 | 149 | 7 |
| | OS | 161 | 5 | 161 | 5 | 163 | 4 | 158 | 5 | 155 | 4 | 153 | 5 | 158 | 5 |

| | | Before | | | | | | | | | |
|---|---|---|---|---|---|---|---|---|---|---|---|
| | | first injection | | 13 Jan. 2019 | | 9 Feb. 2019 | | 13 Feb. 2019 | | 26 Mar. 2019 | |
| | | OCT | IOP | OCT | IOP | OCT | IOP | OCT | IOP | OCT | IOP |
| | | [nm] | [mmHg] | [nm] | [mmHg] | [nm] | [mmHg] | [nm] | [mmHg] | [nm] | [mmHg] |
| 2 | OD | 149 | 4 | 145 | 6 | 153 | 6 | 149 | 4 | 155 | 7 |
| | OS | 155 | 6 | 158 | 10 | 160 | 9 | 161 | 3 | 161 | 6 |
| 3 | OD | 147 | 10 | 150 | 12 | 153 | 9 | 155 | 12 | 159 | 6 |
| | OS | 158 | 7 | 163 | 10 | 160 | 7 | 159 | 9 | 158 | 10 |
| 4 | OD | 159 | 6 | 157 | 3 | 160 | 6 | 162 | 2 | 164 | 6 |
| | OS | 154 | 5 | | 7 | 153 | 4 | 155 | 5 | 154 | 6 |

### Example 4—Second Toxicity Test of Intravitreally Administered Amphiregulin (Member of the EGF-Family) (Guinea Pigs)

Purpose: The purpose of the study was to examine the safety of intravitreally injected epidermal growth factor (EGF).

Methods and Material: Two to three-week old guinea pigs were divided into four groups:

animals that received an intravitreal administration of amphiregulin (dose: 10 ng in 5 μl) into one eye and an intravitreal administration of vehicle into the contralateral eye (n=4),

animals that received an intravitreal administration of amphiregulin (dose: 10 ng in 5 μl) into both eyes (n=4),

an animal that received intravitreal administration of amphiregulin (dose: 10 ng in 5 μl) into one eye with the contralateral eye left untreated (n=1), and

animals with both eyes left intact (n=2).

All animals were treated in accordance with the ARVO (association for Research in Vision and Ophthalmology) Statement for the Use of Animals in Ophthalmic and Vision Research and the EC Directive 86/609/EEC for animal experiments. The animals were housed at a constant temperature (22±1° C.) and in a light-controlled environment (lights on from 7 am to 7 pm) with ad libitum access to food and water.

All animals with injections received two intravitreal injections at an interval of ten days.

At each time point, the eyes were inspected for signs of toxicity. Ten days after the second injection, the animals were sacrificed by an overdose of anesthetics. Ocular biometry was performed at baseline, prior to each injection, and prior to the sacrifice of animals. The sonographic ocular biometry was performed using Aviso, The Ultrasound Platform (Quantel Medical, France) under general anesthesia, on the follow-up days 15, 25, 35 and prior the sacrifice on day 45.

After the sacrifice, the eyes of the animals were removed, post-fixed overnight in 4% paraformaldehyde, and embedded in optimal cutting temperature compound, cryosectioned and immunostained against glial activation marker (glial fibrillary acidic protein), microglial cell marker (lba-1), native guinea pig amphiregulin, and injected amphiregulin; or they were embedded in paraffin, sectioned and stained with hematoxylin and eosin for general histologic examination and histomorphometric retinal thickness measurements. In addition, the tissue was stained for apoptotic cell death detection applying the TUNEL method.

Results: The animals from the four different groups did not differ in their weight at any time point analyzed. No significant increase in TUNEL positive cell numbers, retinal gliosis or significant increase in microglial infiltration were observed in the eyes injected with amphiregulin as compared to the eyes injected with PBS or the untouched eyes. Similarly, amphiregulin immunohistochemical expression in the retina or lens did not differ significantly between the different groups.

### Example 5—Effects of EGF and Anti-EGF on the Proliferation and Migration of EGF Cells

Purpose: It was the purpose of the study to assess the effect of EGF on retinal pigment epithelium (RPE) cells in cell culture.

Materials and Methods: The human ARPE-19 cell line was cultured in a standardized culture medium and incubated at 37° C. under 5% CO₂. Recombinant human EGF was added in concentrations of 0.1 μg/L, 10 μg/L, and 100 μg/L, and the EGF antibody into concentrations of 0.01 mg/L, 1.0 mg/L, and 10 mg/L. As control we used a PBS solution (phosphate buffered solution). We examined the RPE cell proliferation using of a scanning spectrophotometer. In a second step, we examined the cell migration by a scratch assay.

Results: In a dose-dependent manner, the ARPE-19 cell proliferation and migration increased with higher concentrations of EGF and decreased with higher concentrations of EGF-antibody.

Many modifications and other embodiments of the invention set forth herein will come to mind to the one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

### REFERENCES

Berasain C, Avila M A. Amphiregulin. Semin Cell Dev Biol. 2014; 28:31-41.

Binder S, Krebs I, Ralf-Dieter H, et al. Outcome of transplantation of autologous retinal pigment epithelium in age-related macular degeneration: a prospective trial. Invest Ophthalmol Vis Sci 2004; 45(11): 4151-4160

Cooper, Mark; Dimitria Vranes; Sherif Youssef; Steven A. Stacker; Alison J. Cox; Bishoy Rizkalla; David J. Casley; Leon A. Bach; Darren J. Kelly; Richard E. Gilbert (November 1999). “Increased Renal Expression of Vascular Endothelial Growth Factor (VEGF) and Its Receptor VEGFR-2 in Experimental Diabetes” (PDF). Diabetes. 48 (11): 2229-2239.

Donaldson M J and Pulido J S. Treatment of nonexudative (dry) age-related macular degeneration. Curr Opin Ophthalmol 2006; 17(3): 267-274.

Defoe D M, Grindstaff R D. Epidermal growth factor stimulation of RPE cell survival: contribution of phosphatidylinositol 3-kinase and mitogen-activated protein kinase pathways. Exp Eye Res. 2004 July; 79(1):51-9

Downward J, Parker P, Waterfield M D (1984). “Autophosphorylation sites on the epidermal growth factor receptor”. Nature. 311 (5985): 483-5.

Dreux A C, Lamb D J, Modjtahedi H, Ferns G A. The epidermal growth factor receptors and their family of ligands: their putative role in atherogenesis. Atherosclerosis 2006; 186 (1):38-53.

Fronk A H, Vargis E. Methods for culturing retinal pigment epithelial cells: a review of current protocols and future recommendations. J Tissue Eng. 2016 Jul. 12; 7

Gagliardi G, Ben M'Barek K, Goureau O. Photoreceptor cell replacement in macular degeneration and retinitis pigmentosa: A pluripotent stem cell-based approach. Prog Retin Eye Res. 2019; 71:1-25.

Herbst R S (2004). “Review of epidermal growth factor receptor biology”. International Journal of Radiation Oncology, Biology, Physics. 59 (2 Suppl): 21-6.

Holekamp N, Wykoff C C, Schmitz-Valckenberg S, Monés J, Souied E H, Lin H, Rabena M D, Cantrell R A, Henry E C, Tang F, Swaminathan B, Martin J, Ferrara D, Staurenghi G. Natural History of Geographic Atrophy Secondary to Age-Related Macular Degeneration: Results from the Prospective Proxima A and B Clinical Trials. Ophthalmology. 2019 Dec. 14. pii: S0161-6420(19)32353-X. doi: 10.1016/j.ophtha.2019.12.009. [Epub ahead of print]

Hu J, He T C and Li F. For your eyes only: harnessing human embryonic stem cell-derived retinal pigment epithelial cells to improve impaired vision. Genes Dis 2015; 2(4): 293-294.

Kandasamy R, Wickremasinghe S, Guymer R. New treatment modalities for geographic atrophy. Asia Pac J Ophthalmol (Phila). 2017; 6(6):508-513.

Koirala A, Conley S M and Naash M I. A review of therapeutic prospects of non-viral gene therapy in the retinal pigment epithelium. Biomaterials 2013; 34: 7158-7167.

Laude A, Tan L E, Wilson C G, Lascaratos G, Elashry M, Aslam T, Patton N, Dhillon B. Intravitreal therapy for neovascular age-related macular degeneration and inter-individual variations in vitreous pharmacokinetics. Prog Retin Eye Res. 2010; 29:466-475.

Mehta H, Tufail A, Daien V, Lee A Y, Nguyen V, Ozturk M, Barthelmes D, Gillies M C. Real-world outcomes in patients with neovascular age-related macular degeneration treated with intravitreal vascular endothelial growth factor inhibitors. Prog Retin Eye Res. 2018; 65:127-146.

Merle B M J, Colijn J M, Cougnard-Grégoire A, de Koning-Backus A P M, Delyfer M N, Kiefte-de Jong J C, Meester-Smoor M, Féart C, Verzijden T, Samieri C, Franco O H, Korobelnik J F, Klaver C C W, Delcourt C; EYE-RISK Consortium. Mediterranean Diet and Incidence of Advanced Age-Related Macular Degeneration: The EYE-RISK Consortium. Ophthalmology. 2019; 126:381-390.

Nazari H, Zhang L, Zhu D, Chader G J, Falabella P, Stefanini F, Rowland T, Clegg D O, Kashani A H, Hinton D R, Humayun M S. Stem cell based therapies for age-related macular degeneration: The promises and the challenges. Prog Retin Eye Res. 2015; 48:1-39.

Oda K, Matsuoka Y, Funahashi A, Kitano H (2005). “A comprehensive pathway map of epidermal growth factor receptor signaling”. Molecular Systems Biology. 1 (1): E1—E17.

Palmer, Biff F.; Clegg, Deborah J. (2014). “Oxygen sensing and metabolic homeostasis”. Molecular and Cellular Endocrinology. 397 (1-2): 51-57.

Peng C H, Cherng J Y, Chiou G Y, et al. Delivery of Oct4 and SirT1 with cationic polyurethanes-short branch PEI to aged retinal pigment epithelium. Biomaterials 2011; 32: 9077-9088

Singh S, Woerly S and Mclaughlin B J. Natural and artificial substrates for retinal pigment epithelial monolayer transplantation. Biomaterials 2001; 22: 3337-3343. 91

Spraul C W, Kaven C, Lang G K, Lang G E. Effect of growth factors on bovine retinal pigment epithelial cell migration and proliferation. Ophthalmic Res. 2004 May-June; 36(3):166-71

Tode J, Richert E, Koinzer S, Klettner A, von der Burchard C, Brinkmann R, Lucius R, Roider J. Selective Retina Therapy Reduces Bruch's Membrane Thickness and Retinal Pigment Epithelium Pathology in Age-Related Macular Degeneration Mouse Models. Transl Vis Sci Technol. 2019; 8:11.

Vavvas D G, Small K W, Awh C C, Zanke B W, Tibshirani R J, Kustra R. CFH and ARMS2 genetic risk determines progression to neovascular age-related macular degeneration after antioxidant and zinc supplementation. Proc Natl Acad Sci U S A. 2018; 115:E696-E704.

Yan F, Hui Y N, Li Y J, Guo C M, Meng H. Epidermal growth factor receptor in cultured human retinal pigment epithelial cells. Ophthalmologica. 2007; 221(4):244-50

Yarden Y, Schlessinger J (March 1987). “Epidermal growth factor induces rapid, reversible aggregation of the purified epidermal growth factor receptor”. Biochemistry. 26 (5): 1443-51.

Yuan Z, Ding S, Yan M, et al. Variability of miRNA expression during the differentiation of human embryonic stem cells into retinal pigment epithelial cells. Gene 2015; 569: 239-249.

Zeng, F., Singh, A. B., & Harris, R. C. (2009). The role of the EGF family of ligands and receptors in renal development, physiology and pathophysiology. Experimental cell research, 315(4), 602-610. https://doi.org/10.1016/j.yexcr.2008.08.005

1. A method for treating a retinal pigment epithelium (RPE) damage associated disease in a patient, wherein the method comprises administering to the patient a pharmaceutical composition comprising an epidermal growth factor receptor (EGFR) agonist, wherein the EGFR agonist comprises the EGF family consensus amino acid sequence CX₇CX₄₋₅CX₁₀₋₁₃CXCX₅GXRC (SEQ ID NO: 1), wherein X is any proteogenic amino acid, wherein the RPE damage associated disease is selected from age-related macular degeneration (AMD), retinitis pigmentosa, cone-rod dystrophies or cone dystrophies, polypoidal choroidal vasculopathy, and Stargardt's disease.

2. The method according to claim 1, wherein the EGFR agonist comprises an amino acid sequence with an identity to a member of the EGF family of at least 80%, at least 90%, at least 95%, at least 98% or 100%.

3. The method according to claim 2, wherein the member of the EGF family is selected from EGF, heparin binding EGF (HB-EGF), transforming growth factor-α (TGF-α), amphiregulin (AR), epiregulin (EPR), epigen, betacellulin (BTC), neuregulin-1 (NRG1), neuregulin-2 (NRG2) neuregulin-3 (NRG3), and neuregulin-4 (NRG4) or a fragment thereof.

4. The method according to claim 3, wherein the member of the EGF family is human.

5. The method according to claim 1, wherein the member of the EGFR agonist comprises an amino acid sequence selected from SEQ ID NO: 3, SEQ ID NO: 5, SEQ ID NO: 7, SEQ ID NO: 9, SEQ ID NO: 11, SEQ ID NO: 13, SEQ ID NO: 15, SEQ ID NO: 17, SEQ ID NO: 19, and SEQ ID NO: 21.

6. The method according to claim 4, wherein the EGFR agonist comprises EGF according to SEQ ID NO: 1 or a fragment thereof, preferably consists of Pro-EGF, EGF or a fragment thereof.

7. The method according to claim 4, wherein the EGFR agonist comprises amphiregulin or a fragment thereof, preferably consists pro-amphiregulin, amphiregulin or a fragment thereof.

8. The method according to claim 1, wherein the EGFR agonist is recombinantly expressed in bacteria, such as E. coli, or eukaryotic cells, such as a mammalian, insect, plant, or fungal cell.

9. The method according to claim 1, wherein said method is a therapeutic of prophylactic treatment.

10. The method according to claim 1, wherein said method comprises administering the EGFR agonist intravitreally, preferably in the temporal inferior quadrant of the eye more preferably through the conjunctiva, sclera and pars plana into the vitreous cavity.

11. The method according to claim 1, wherein said method comprises administering the pharmaceutical composition in a distance of 1 mm to 6 mm from the limbus, more preferably in a distance of 2 mm to 5 mm, most preferably in a distance of 3 mm to 4 mm posterior to the corneal limbus.

12. The method according to claim 10, wherein said method comprises administering the EGFR agonist in a dosage in the range of 0.30 μg to 600 μg per eye, preferably in the range of 0.50 μg to 300 μg, more preferably in the range of 0.50 μg to 100 μg.

13. The method according to claim 1, wherein said method comprises administering the pharmaceutical composition multiple times, with a time interval between the administrations in the range of 3 days to 12 weeks, preferably in the range of 2 weeks to 8 weeks, more preferably 3 weeks to 5 weeks.

14. The method according to claim 1, wherein the pharmaceutical composition is a solution or a lyophilisate.

15. The method according to claim 1, wherein said method comprises the treatment of a human.

16. The method according to claim 1, wherein the pharmaceutical composition does not comprise stem cells.


SEQUENCE LISTING (21 sequences)

SEQ ID NO: 1 - protein, 40 aa, Homo sapiens
  MISC_FEATURE (2)..(8): Xaa can be any amino acid
  MISC_FEATURE (10)..(13): Xaa may be any amino acid
  MISC_FEATURE (14)..(14): Xaa may be any amino acid or absent
  MISC_FEATURE (16)..(25): Xaa may be any amino acid
  MISC_FEATURE (26)..(28): Xaa may be any amino acid or absent
  MISC_FEATURE (30)..(30): Xaa may be any amino acid
  MISC_FEATURE (32)..(36): Xaa may be any amino acid
  MISC_FEATURE (38)..(38): Xaa may be any amino acid
CXXXXXXXCXXXXXCXXXXXXXXXXXXXCXCXXXXXGXRC

SEQ ID NO: 2 - protein, 53 aa, Homo Sapiens
NSDSECPLSHDGYCLHDGVCMYIEALDKYACNCVVGYIGERCQYRDLKWWELR

SEQ ID NO: 3 - protein, 37 aa, Homo Sapiens
CPLSHDGYCLHDGVCMYIEALDKYACNCVVGYIGERC

SEQ ID NO: 4 - protein, 87 aa, Homo sapiens
SVRVEQVVKPPQNKTESENTSDKPKRKKKGGKNGKNRRNRKKKNPCNAEFQNFCIHGECKYIEHLEAVTCKCQQEYFGERCGEKSMK

SEQ ID NO: 5 - protein, 36 aa, Homo sapiens
CNAEFQNFCIHGECKYIEHLEAVTCKCQQEYFGERC

SEQ ID NO: 6 - protein, 85 aa, Homo sapiens
LQEADLDLLRVTLSSKPQALATPNKEEHGKRKKKGKGLGKKRDPCLRKYKDFCIHGECKYVKELRAPSCICHPGYHGERCHGLSL

SEQ ID NO: 7 - protein, 36 aa, Homo sapiens
CLRKYKDFCIHGECKYVKELRAPSCICHPGYHGERC

SEQ ID NO: 8 - protein, 50 aa, Homo sapiens
VVSHFNDCPDSHTQFCFHGTCRFLVQEDKPACVCHSGYVGARCEHADLLA

SEQ ID NO: 9 - protein, 36 aa, Homo sapiens
CPDSHTQFCFHGTCRFLVQEDKPACVCHSGYVGARC

SEQ ID NO: 10 - protein, 132 aa, Homo sapiens
AAVTVTPPITAQQGNWTVNKTEADNIEGPIALKFSHLCLEDHNSYCINGACAFHHELEKAICRCFTGYTGERCEHLTLTSYAVDSYEKYIAIGIGVGLLLSGFLVIFYCYIRKRCLKLKSPYNVCSGERRPL

SEQ ID NO: 11 - protein, 36 aa, Homo sapiens
CLEDHNSYCINGACAFHHELEKAICRCFTGYTGERC

SEQ ID NO: 12 - protein, 80 aa, Homo sapiens
DGNSTRSPETNGLLCGDPEENCAATTTQSKRKGHFSRCPKQYKHYCIKGRCRFVVAEQTPSCVCDEGYIGARCERVDLFY

SEQ ID NO: 13 - protein, 36 aa, Homo sapiens
CPKQYKHYCIKGRCRFVVAEQTPSCVCDEGYIGARC

SEQ ID NO: 14 - protein, 222 aa, Homo sapiens
SGKKPESAAGSQSPALPPRLKEMKSQESAAGSKLVLRCETSSEYSSLRFKWFKNGNELNRKNKPQNIKIQKKPGKSELRINKASLADSGEYMCKVISKLGNDSASANITIVESNEIITGMPASTEGAYVSSESPIRISVSTEGANTSSSTSTSTTGTSHLVKCAEKEKTFCVNGGECFMVKDLSNPSRYLCKCQPGFTGARCTENVPMKVQNQEKAEELYQK

SEQ ID NO: 15 - protein, 40 aa, Homo sapiens
CAEKEKTFCVNGGECFMVKDLSNPSRYLCKCQPGFTGARC

SEQ ID NO: 16 - protein, 293 aa, Homo sapiens
CYSPSLKSVQDQAYKAPVVVEGKVQGLVPAGGSSSNSTREPPASGRVALVKVLDKWPLRSGGLQREQVISVGSCVPLERNQRYIFFLEPTEQPLVFKTAFAPLDTNGKNLKKEVGKILCTDCATRPKLKKMKSQTGQVGEKQSLKCEAAAGNPQPSYRWFKDGKELNRSRDIRIKYGNGRKNSRLQFNKVKVEDAGEYVCEAENILGKDTVRGRLYVNSVSTTLSSWSGHARKCNETAKSYCVNGGVCYYIEGINQLSCKCPNGFFGQRCLEKLPLRLYMPDPKQKAEELYQK

SEQ ID NO: 17 - protein, 37 aa, Homo sapiens
CNETAKSYCVNGGVCYYIEGINQLSCKCPNGFFGQRC

SEQ ID NO: 18 - protein, 358 aa, Homo sapiens
MSEGAAAASPPGAASAAAASAEEGTAAAAAAAAAGGGPDGGGEGAAEPPRELRCSDCIVWNRQQTWLCVVPLFIGFIGLGLSLMLLKWIVVGSVKEYVPTDLVDSKGMGQDPFFLSKPSSFPKAMETTTTTTSTTSPATPSAGGAASSRTPNRISTRLTTITRAPTRFPGHRVPIRASPRSTTARNTAAPATVPSTTAPFFSSSTLGSRPPVPGTPSTQAMPSWPTAAYATSSYLHDSTPSWTLSPFQDAASSSSSSSSSATTTTPETSSPKFHTTTYSTERSEHFKPCRDKDLAYCLNDGECFVIETLTGSHKHCRCKEGYQGVRCDQFLPKTDSILSDPTDHLGIEFMESEEVYQR

SEQ ID NO: 19 - protein, 39 aa, Homo sapiens
CRDKDLAYCLNDGECFVIETLTGSHKHCRCKEGYQGVRC

SEQ ID NO: 20 - protein, 61 aa, Homo sapiens
MPTDHEEPCGPSHKSFCLNGGLCYVIPTIPSPFCRCVENYTGARCEEVFLPGSSIQTKSNL

SEQ ID NO: 21 - protein, 37 aa, Homo sapiens
CGPSHKSFCLNGGLCYVIPTIPSPFCRCVENYTGARC